(19) United States
(12) Patent Application Publication
(10) Pub. No.: US 2005/0096652 A1
(43) Pub. Date: May 5, 2005

(54) Integral flexible spine stabilization device and method

(76) Inventor: Charles V. Burton, Excelsior, MN (US)

Correspondence Address: OPPENHEIMER WOLFF & DONNELLY LLP, 45 SOUTH SEVENTH STREET, SUITE 3300, MINNEAPOLIS, MN, 55402, US

(21) Appl. No.: 10/698,650

(22) Filed: Oct. 31, 2003

(51) Int. Cl. A61F 2/30; A61B 17/56

(52) U.S. Cl. 606/61

(57) ABSTRACT

Provided are a device and a method for stabilizing two or more vertebrae by joining the vertebrae with a flexible device. The flexible device is of unitary construction and includes anchor posts constructed and arranged for insertion into holes drilled into the vertebrae, preferably after removing all or a portion of the pedicles from the target vertebrae. Removal of the pedicles provides an acceptable fastening site, prevents interference with the device by the pedicles, and provides native donor bone tissue, thereby obviating the need for a second surgery to harvest bone tissue from a different site, such as the hip.

This application provides a method and device useable to flexibly connect at least two vertebrae together, thereby increasing the stability of the vertebral column, and also allow for the adequate decompression of pinched nerves. The present invention improves upon related devices and methods, invented by the Applicant, and fully described in U.S. Pat. No. 4,743,260 and U.S. Pat. No. 5,282,863. The contents of these patents are incorporated by reference herein in their entireties.

### BACKGROUND OF THE INVENTION

Stabilization of the spinal vertebrae has traditionally been accomplished using fusion, a long-practiced procedure involving the permanent and rigid fixation of one or more spinal segments. Each spinal segment includes two adjacent vertebrae, their posterior bony elements, an intervertebral disc between the two vertebrae, ligaments, and two facet joint capsules. Stabilizing two or more vertebrae together is done in an attempt to improve spinal stability and prevent the occurrence of “slipped disks” and other related spinal problems, and to treat disability due to pain and/or pinched nerves.

Established methods of rigid spinal fusion have significant drawbacks. When two or more vertebrae are totally fused together, and thus immobilized, the movement and stresses that the fused vertebrae once provided and absorbed are transferred to the remaining mobile vertebrae. Especially affected are those vertebrae immediately adjacent the fused section. These adjacent vertebrae are subject to inordinately high stress and degeneration often leading to the need for additional surgery. Moreover, in the typical rigid L4-S1 fusion, the L3-4 level typically undergoes stress-related degeneration of the disc and zygoapophyseal joints leading to the production of a clinical entity called the “transitional syndrome” often leading to “mechanical-type” pain or actual spinal nerve compression. The stress patterns produced by fusion often also produce additional clinical problems relating to the sacroiliac and hip joints.

Safe, effective and more physiologic long-term stabilization has been difficult to accomplish. When spine fusions involve mechanical instrumentation, significant adverse forces are directly aimed at the supportive sites whether they are bone screws, hooks, or the like. This phenomenon usually wears away at the relatively soft bone matter, resulting a loosening of the attachment points for the implanted hardware and a resulting loss of support by this instrumentation. Thus, fusions involving instrumentation are often carried out in conjunction with bone fusions so that as the instrumentation loosens and fails, support can be maintained by growth of the bony counterpart. These combined procedures involve extensive surgery, substantial blood loss and high cost very often creating more problems for the patient than those solved. Further, the recovery time for such procedures is significant and debilitating and very often necessitates additional spinal surgery.

Therefore, the present management of spinal instability by instrumented fusion stabilization is not optimal. The fused vertebrae become immobile, eliminating their previous natural ability to move relative to each other. Also, when fusion is combined with added internal fixation, the internal fixation parts are typically metallic. In modern medical practice, the presence of large and dense metallic devices can be a significant liability due to the scattering artifacts generated if imaging, such as MRI, is attempted. At this time MRI imaging is a preferred spinal diagnostic procedure.

In 1973 scientists at the Materials Research Laboratory at Penn State University observed that in some reef building corals their microstructure was characterized by 1) a high degree of uniformity in pore diameter, 2) a pore interconnection diameter similar in size to that of the pore itself, 3) a solid-to-volume ratio of approximately one, and 4) exceptionally high permeability in which each and every pore was interconnected to all other pores. It was recognized that it would be virtually impossible to achieve these structural characteristics by artificial means. Early experiments designed to impregnate the coral with various substances, dissolve the calcium carbonate coral skeleton and then make positive casts with other materials including metal alloys, were successful and the term “the replamineform process” was coined.

In addition to replamineform casting it became evident that the structural characteristics of coral itself could possibly serve as the means for bone ingrowth after a cleansing of organic materials and a chemical conversion from biogenic carbonate to hydroxyapetite.

Subsequent medical studies using coral with a microdiameter (200-250 microns) similar to that of the normal human bone Haversian canals (200-400 microns) were conducted by a number of physicians in the United States. Drs. Vert Mooney and David Selby in Dallas, Tex. performed 40 cases of bilateral spinal bone fusion in which one side was coral provided by Interpore International of Irvine, Calif., and the other side autogenous bone. These studies did not demonstrate significant ingrowth of osteoblasts into the coral and these observations led to the conclusion that coral showed little value in this regard. Since this work most clinicians have almost exclusively used autogenous or donor derived homologous bone.

The applicant has overcome some of the aforementioned difficulties with the devices and methods described in his U.S. Pat. Nos. 4,743,260 and 5,282,863. These patents provide a device and method for stabilizing a portion of a vertebral column without fusion using a stabilization device made of a biocompatible, non-metallic material. The device includes an anchor means and a stabilization means. The anchor means includes an upper shank portion and a lower threaded portion cooperatively connected to the shank portion. The shank portion is self-tapping such that it may be screwed into the pedicle of a vertebra. The stabilization means includes a first stabilization element having first and second openings and a second stabilization element having first and second openings. The openings are arranged to be placed over the upper shank portions of the anchor means. The method involves making an incision to expose the first and second vertebrae to be stabilized. The posterior vertebral elements of each of the vertebrae to be stabilized are removed, leaving first and second pedicles of the first vertebra and first and second pedicles of the second vertebra. A bone screw is secured into at least one of the pedicles of the first and second vertebrae. The stabilization means is cooperatively connected between the bone screw of one of the first pedicles of the first vertebra to the bone screw of one of the first pedicles of the second vertebra.

The device and method taught by U.S. Pat. Nos. 4,743,260 and 5,282,863 effectively provide a desired degree of flexibility. However, self-tapping bone screws, though widely used for a variety of applications, typically struggle to provide both the strength sufficient enough to drive themselves into bone, and the porosity necessary to promote ingrowth. Additionally, once the bone screws are anchored into the vertebral column, the stabilization means must be attached to the exposed ends of the screws with fasteners. Though the fasteners taught by these patents are effective, it would be advantageous to provide a device that obviates the need for fasteners and self-tapping bone screws.

### BRIEF SUMMARY OF THE INVENTION

The present invention addresses the problems associated with the prior art devices and methods for treating spinal instability, and provides for unitary flexible stabilization, rather than component flexible stabilization; meaning the spine is supported yet allowed a degree of natural movement or flex. This is accomplished with a minimal amount of metallic components. Additionally, the present invention may allow cellular infiltration by fibroblasts, osteoblasts, reticulation, or other cellular components, to occur. Included in the present invention is a device and method for stabilizing a portion of a vertebral column without fusion. In the preferred form the stabilization device of the present invention is substantially completely or completely made of non-metallic materials (e.g. plastic), which are biocompatible and designed to become integrated with normal body tissues.

The vertebral column is composed of segments of adjacent vertebrae, with the first vertebra being adjacent the second vertebra. Each vertebra has a posterior vertebral element which, when removed, leaves first and second pedicles on each of the vertebra. The present device includes strong nonmetallic anchor means for securing the stabilization means to at least one of the pedicles of the first and second vertebrae and means for cooperatively connecting the stabilization means to the anchor means. In a preferred embodiment, the device further includes a means for locking the stabilization means to the anchor means.

In one embodiment, the anchor means includes an upper shank portion and a lower threaded portion having a screw thread. The lower portion is cooperatively connected to the shank portion. The screw thread has segmented areas, wherein a rotary force may be applied to the shank portion whereby the threaded portion is driven into and secured into the pedicles. The screw thread may have segmented areas, wherein after a period of time the vertebra's bony re-growth encompasses the segmented areas to further secure the threaded portion to the pedicles.

Preferably, the stabilization means includes a first stabilization element having first and second openings and a second stabilization element having first and second openings. The anchor means comprises four support members such that one of the support members is secured to each of the pedicles, wherein each of the openings is adapted for being positioned around one of the support members. The stabilization elements may then be aligned along the four support members in either a generally parallel relationship or in an X shape. Further, a cross support member may be used to further connect the first stabilization element to second stabilization element.

In a more preferred embodiment, the anchor means and the stabilization means are of one-piece unitary construction. The anchor means has an upper portion and a lower portion. The upper portion is integral with the stabilization means. The lower portion forms an elongate post, insertable into a hole drilled into the pedicle of a vertebra. To promote ingrowth, the post is porous. Additionally, the post is smaller than the diameter of the drilled hole, leaving sufficient room for a desired quantity of a slurry paste formed from ground bone tissue that includes stem cells, thereby encouraging an ingrowth of bone tissue into the post. In an alternative embodiment the post is hollow forming a channel or lumen therethrough with the outer post areas being porous. This alternative embodiment is designed to promote additional cellular ingrowth.

In a preferred embodiment, the anchoring means and/or stabilization means may be made from a microporous material produced by a suitable process such as a replamineform process, wherein ingrowth by fibroblasts occur. Coral having a microdiameter of between 190-1200 microns and preferably between 190-230 microns in diameter is used and poritic corals may be used as a model for the replamineform process.

The present invention also includes an activation process, which has been determined to be a key step in the successful integration of microporous system components. The activation can be internal, external or both, and is herein defined as the process of purposefully inducing and supporting local cellular proliferation to promote component integration.

The present invention also comprises a surgical method for stabilizing a portion of a vertebral column without fusion. The method includes making an incision to expose the first and second vertebrae. Then, the posterior vertebral elements of each of the vertebrae to be stabilized are removed, leaving first and second pedicles of the first vertebra and first and second pedicles of the second vertebra. In order to relieve pressure from the nerves stemming from the spinal cord, a medial inferior portion of each of the remaining pedicles is also removed, thereby ensuring the nerve does not abut against the pedicle. Alternatively, if clinical presentation warrants, a full pediclectomy may be performed. A bone screw is secured into at least one of the pedicles of the first and second pedicles of the first and second vertebrae. The bone screw has a threaded bottom portion and an upper shank portion. Next, the stabilization means is cooperatively connected between the bone screw of one of the first pedicles of the first vertebra to the bone screw of one of the first pedicles of the second vertebra. It is understood that screws are not essential, but are a preferred embodiment. Other suitable anchoring means may be used to anchor the stabilization means to pedicles, such as stapling. It is also understood that multiple segments may be stabilized.

A preferred embodiment of a surgical method for stabilizing a portion of a vertebral column without fusion includes making an incision to expose the first and second vertebrae. Then, the posterior vertebral elements of each of the vertebrae to be stabilized are removed, leaving first and second pedicles of the first vertebra and first and second pedicles of the second vertebra. In order to relieve pressure from the nerves stemming from the spinal cord, a medial inferior portion of each of the remaining pedicles is also removed, thereby ensuring the nerve does not abut against the pedicle. Next, holes are formed in the pedicles spaced apart a predetermined distance to accept porous anchoring posts of a unitarily constructed flexible stabilization device. Alternatively, if clinical presentation warrants, a full pediclectomy may be performed. In such a case, the holes are formed in the vertebral body at the pedicle footprint. This is a particularly strong area because of the fascicular nature of the bone fibres passing up from the vertebral body into the pedicle. If the pedicle is completely removed, i.e. flush with the vertebral body, this sit is still highly reinforced and ideal for anchoring. In both case, optimally the removed pedicles are used to create an activation slurry of milled bone and autogenous stem cells and/or bone morphogenic materials. An appropriate quantity of the slurry is placed in the holes along with the anchoring posts of the device. A temporary bond is created between the device and the vertebrae using a polymer or cement, thereby securing the device to the vertebrae while ingrowth is promoted.

### BRIEF DESCRIPTION OF THE DRAWINGS

FIG. 1 is a perspective view showing one embodiment of the present invention in a partially exploded perspective view;

FIG. 2 is a top-plan view of the present invention shown in FIG. 1;

FIG. 3 is a perspective view of the bone screw of the present invention;

FIG. 4 is a cross-sectional view taken generally along the lines 4-4 of FIG. 3;

FIG. 5 is a cross-sectional view of one embodiment of the locking cap as shown in FIG. 1;

FIG. 6 is a perspective view of a second embodiment of a locking cap;

FIG. 7 is a cross-sectional view taken generally along the lines 7-7 of FIG. 6;

FIG. 8 is a partial cross-sectional view showing a bone screw being inserted into a vertebra according to the present invention;

FIG. 9 is a side elevation of a vertebra before the posterior vertebral elements are removed according to the present invention;

FIG. 10 is perspective view of a second embodiment of a bone screw;

FIG. 11 is a perspective view of a second embodiment of a stabilization element;

FIG. 12 is a perspective view of a unitary embodiment of a stabilization device with integral anchoring posts;

FIG. 13 is a plan view of a unitary embodiment of a stabilization device with integral anchoring posts; and

FIG. 14 is a perspective view of a unitary embodiment of a stabilization device with integral anchoring posts.

### DETAILED DESCRIPTION OF THE INVENTION

Referring to the figures, wherein like numerals represent like parts throughout the several views, there is generally illustrated a device 10 useable for stabilizing at least a portion of a vertebral column. In FIG. 1, a first vertebra 11 and second vertebra 12 are shown. It should be understood that when the terms “first and second” vertebrae are used in this application, reference is being made only to vertebrae that are adjacent, and not to any specific vertebrae along the vertebral column. Each vertebra has a posterior element, designated as 13 shown in FIG. 9. When the posterior element 13 is removed from the vertebra, a first and second vertebral pedicle is exposed in each vertebra. FIG. 1 shows the first vertebral pedicle 11a and the second vertebral pedicle 11b of the first vertebra 11 and the first vertebral pedicle 12a and the second vertebral pedicle 12b of the second vertebra 12. If screws are used as anchoring devices, each of the pedicles (11a, 11b, 12a or 12b) that are to be utilized have a hole 14 drilled into the pedicle.

A bone screw, generally designated as 15, has an upper shank portion 16, which in a preferred embodiment has a square cross section to allow for efficient insertion and removal. A lower threaded portion 17 is cooperatively connected to the upper shank portion 16. The threaded portion 17 has an inner shaft 17a and a threaded member 17b. The threaded member 17b is a continuous helical member having segmented areas 17c. The threaded member 17 provides a wide flange to give a deep bite when secured in the vertebra. While the segmented areas 17c are shown in FIG. 4 as being pie-shaped, and having three per one complete circumference, it is understood that any suitable shape or number of segmented areas may be utilized. The inner shaft 17a and threaded member 17b have a generally consistent diameter throughout, except for a taper towards a point at their bottom end. Preferably, the diameter of the hole 14 is slightly smaller than the diameter of the inner shaft 17a.

A stabilization element 18, having a first end 18a and a second end 18b has two openings 19 adapted to being placed over the upper shank 16 of the bone screw 15. A spacer 20 may also be used if the height of the pedicle is not sufficient. The spacer 20 can be inserted between the pedicle and stabilization element 18 to avoid compression of exiting spinal nerves. In one embodiment, the element 18 is a flat strip. However, it is understood that other suitable shapes such as an hourglass shape or dumbbell shape may be used.

A locking cap 21, having an opening 21a has a generally downwardly depending circumferential member 21b that locks the rod 18 in place. A second embodiment of a locking cap is shown in FIGS. 6 and 7. The locking cap 22 is similar to the locking cap 21 in that it has an opening 22a and a generally downwardly depending circumferential member 22b, similar to locking cap 21. However, in addition, there are two skirt members 22c. As will be more fully discussed hereinafter, the skirt members 22c limit parallelogram movement between the two stabilization elements 18 when they are in position.

The material that the stabilization elements 18 and screws 15 are made of must provide sufficient strength, be biocompatible, and preferably non-metallic. Some non-reinforced biocompatible plastic polymers indicate a tendency to crack, fissure or shear with repeated flex or stress. A preferred material is a two-phase biocompatible plastic so as to provide adequate strength. Internal reinforcement with dissimilar polymers or filaments provide increased strength over that of a single phase plastic, but as the internal diameter of the stabilization element 18 decreases, practical fabrications become a problem. Therefore, it is preferred that internal fibers are used for reinforcement. It is desired that the material used have sufficient strength and flexibility, as well as being biocompatible.

It is known that many fibers have been tested in polymers and many lightweight and remarkably strong materials have resulted. A most innovative reinforced two-phase material to date has been created for aeronautical use. Although many of these systems are attractive for their physical qualities, they are also marginal or clearly unacceptable from the standpoint of being biocompatible. It has been found that carbon fiber reinforced plastic yields adequate strength for constructing the stabilization elements 18. However, it is sometimes preferable that the screws be made of a still stronger material so as to prevent the screws from being sheared off by the stress of the system. One such material that has been found adequate for the screws 15 is a magnamite graphite fiber or carbon reinforced plastic. The materials used for the stabilization elements 18 and screws 15 are not limited to the above noted plastics, and may also include other suitable solid materials that have the above-noted properties. Also, porous material forms in which the porosity is controlled by the replamineform process may be utilized. Polymers such as silicone, polyethylene, nylon, vinyl, methylmethacyrate, dacrons or teflon may be suitable.

Applicant has made a study of small coral specimens and a study of these samples has confirmed the original observations of the Penn State researchers. By matching the physical pore size characteristics of applicant's samples with those reported in the failed medical fusion studies, two conclusions could be drawn. The first is that if coral with larger pore sizes were used (400-500 microns), it might very well be more successful for bone fusion osteoblasts ingrowth. Secondly, the previous medical research on coral, which was considered a failure, actually indicated that there was ingrowth by fibroblasts (rather than osteoblasts). However, such fibroblasts ingrowth is well suited for the flexible system of the present invention. The ingrowth of fibroblasts will further anchor and fix the device 10, but will still allow the device 10 to retain its flexible characteristics.

Further, Applicant has identified poritic coral having 190-230 micron diameter as preferred for the present flexible system rather than other corals, such as goniopera with 230-600 micron size. It is poritic coral that would serve as the replamineform model for making the screws 15 and stabilization elements 18 of the present invention. However, it has been found that pore sizes of from 190-1200 microns are suitable for the present invention.

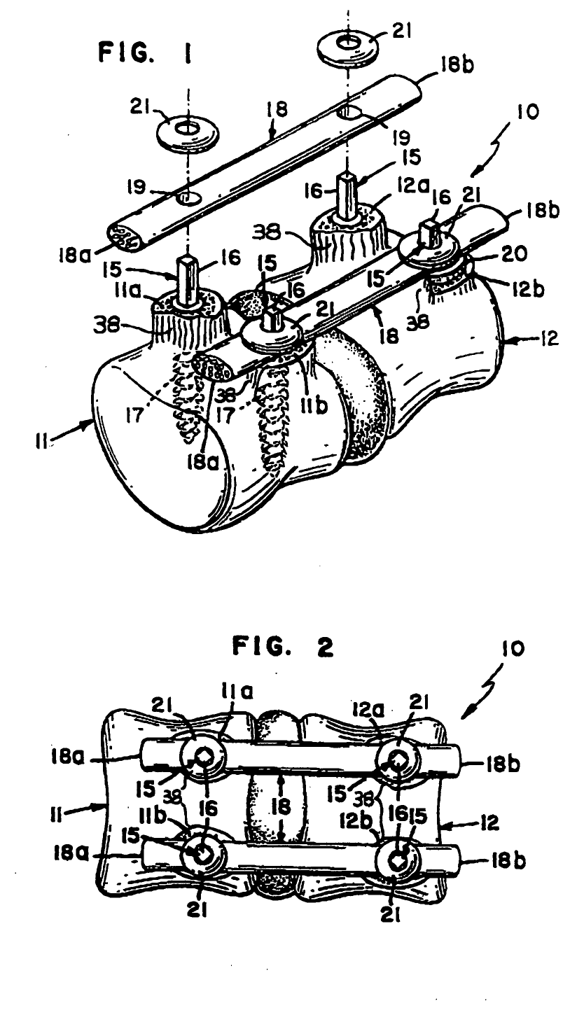

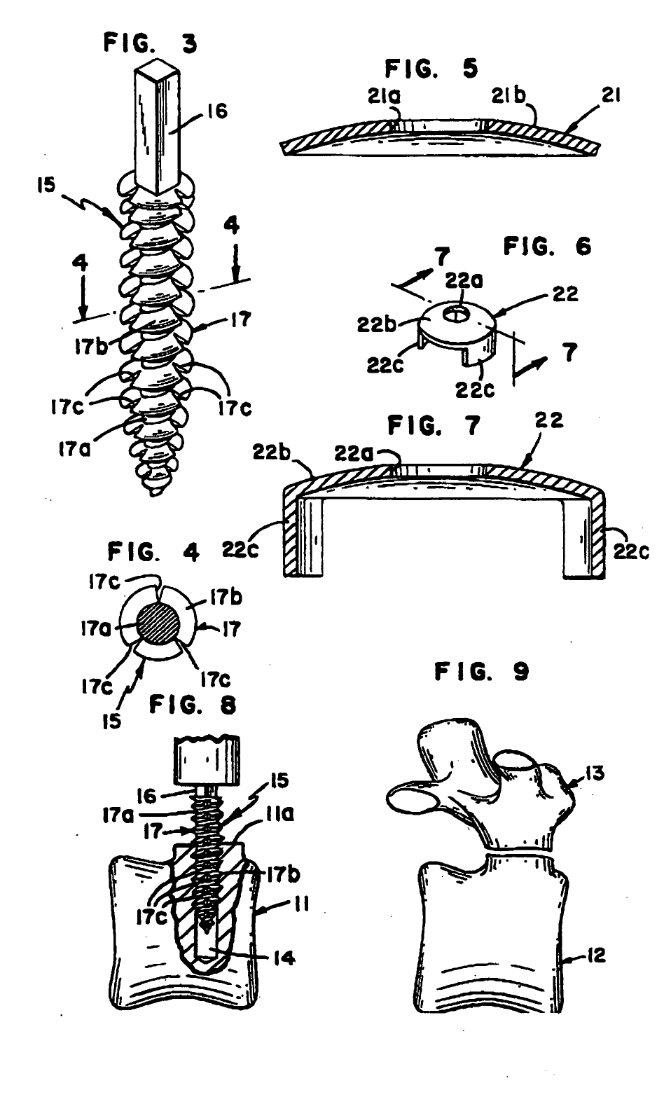

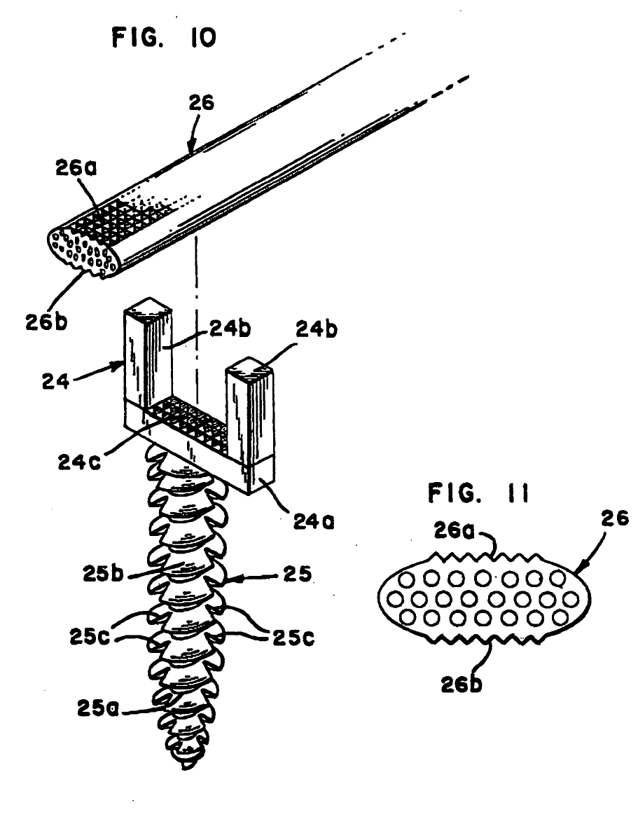

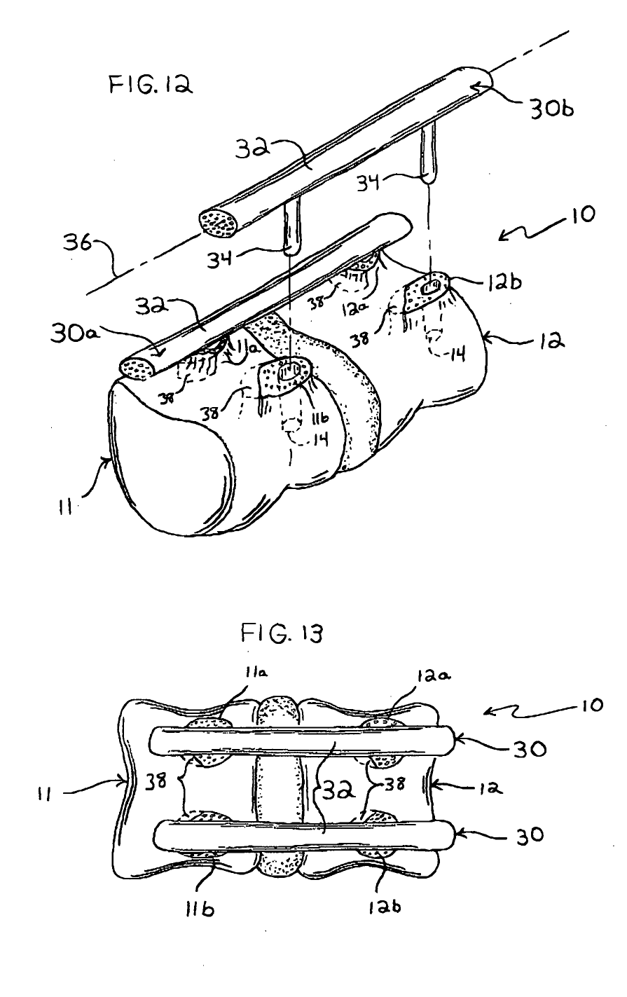

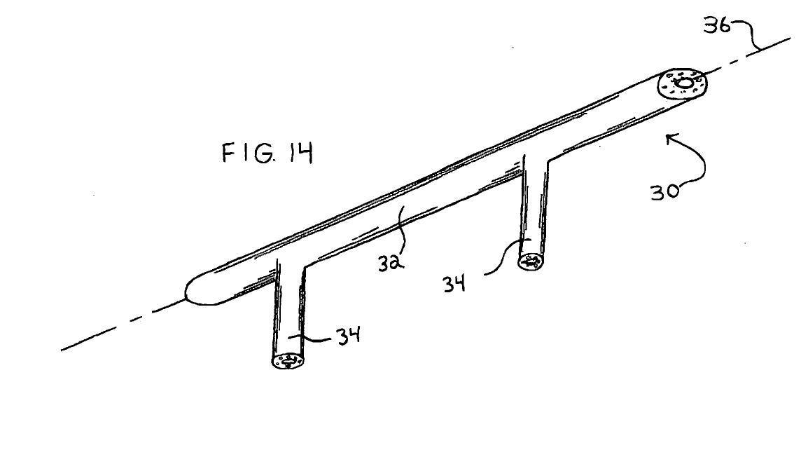

In use, only a limited surgical exposure is necessary to perform the operation necessary to incorporate the present invention. Following the removal of all or part of the posterior vertebral elements 13, the exposed pedicles serve as the anchoring media for the bone screws 15. In addition to removing the posterior vertebral elements 13, medial inferior portions 38 of the remaining pedicles may be removed to alleviate pressure from swollen nerves. The removed portions 38 are shown in FIGS. 1, 12 and 13. The pedicles are drilled by appropriate means to create the hole 14. A driving mechanism is placed over the upper shank 16 and the lower portion 17 is screwed completely into the vertebra. The threaded member 17b serves to stabilize the bone screw 15 and the segmented areas 17c are designed to allow bone growth to further stabilize the screw 15 in place. If only two vertebrae are to be stabilized, it is only necessary for bone screws 15 to be secured into the pedicles 11a, 11b, 12a and 12b . However, if multiple vertebrae are to be stabilized, the corresponding pedicles on the third or subsequent vertebrae also have to be provided with a bone screw 15. The stabilization elements 18 are tapped at the appropriate position and holes 19 are made either by drilling or by a heated rod at the site of the tap. While FIGS. 1 and 2 show that stabilization elements 18 have been placed for a one-level stabilization, the present invention is compatible with stabilizing multiple levels as well as single levels. Further, only one stabilization element 18 may be necessary for some patients. Once the screws 15 are in place, a separator instrument, not shown, may be used to distract the vertebrae, if this desired, and this separation is maintained by the placement of the stabilization elements 18 over the shank 16 of the bone screw 15. As previously mentioned, if the height of the pedicle is not sufficient, a spacer 20 may be inserted on top of the pedicle. The spacer 20 may be constructed from a softer plastic such as polyurethane or silicone. The procedure is then completed by the application of a locking cap, either 21 or 22, at each of the upper shanks 16 and the removal of any excess shank material above the top of the locking cap 21 or 22.

While the device 10 is illustrated in FIGS. 1 and 2 as having a stabilized means having two generally parallel stabilization elements 18, it is understood that the stabilization means may be other suitable forms such as a single stabilization element 18, or two stabilization elements 18 that cross to form an X shape, or two stabilization elements 18 generally parallel having across support member to form an H shape.

The present invention allows for flexibility and because of the flexibility of the system, less disruptive force is applied to the screw 15, or other anchor means, following the application of the system itself.

The flexibility of the device 10 has as its lower limit no flexibility. No flexibility would have the same result as fusion and the device 10 provides for at least some flexibility more than fusion. In the preferred embodiment, the flexibility of the device 10 would stabilize with a degree that substantially equals a normal back. Too much flexibility would render the back non-functional and not stabilize the back. Any stabilization more than that present in the back of the patient before the procedure would be beneficial.

Numerically, the general upper limit of flexibility for the device 10 is shown in the following table for the stabilization means:

| Stabilization | Inch-Pounds for Standard |
|---|---|
| Means Length | Deflection of ½ Inch |
| 1″ | 45 ip. |
| 2″ | 32 ip. |
| 3″ | 23 ip. |

The device 10 is stiff enough to stabilize the vertebral column but flexible enough to permit at least limited normal movement of the vertebral column. The flexibility allows forces on the stabilization elements and anchors to be dissipated throughout, thereby reducing force concentrations.

If locking cap 21 is used, parallelogram movement between the two stabilization elements 18 is possible. That is, there is a possibility of some relative sideways movement of the first vertebra 11 to the second vertebra 12. If locking cap 22 is used, the locking cap 22 will limit the parallelogram movement. This is because the rod 18 is positioned inside of the skirt member 22c such that parallelogram rotation is limited.

In addition to anchoring the stabilization element 18 by means of screws 15, it is also envisioned that other suitable methods of anchoring may be used. One such example would be to staple the stabilization element 18 to the pedicles. Another embodiment would include ribbed tabs molded to the stabilization element 18 as an integral part thereof and would be positioned approximately the same place where the bone screws 15 would be inserted in the previously discussed embodiment. The holes would then be drilled into the pedicles at the appropriate distances and the ribbed tabs could be inserted into the holes and glued in place. The ribbed tabs could be placed at varying distances along the stabilization element in various models and the appropriate length model simply chosen depending upon the spacing between the vertebrae of the patient.

FIGS. 10 and 11 show another embodiment of the present invention wherein it would not be necessary to have an opening in the stabilization rod. The second embodiment of the bone screw 23 has an upper portion 24 and a lower threaded portion 25. The lower threaded portion 25 is similar to the lower threaded section 17 in that the lower portion 25 has a shaft 25a about which is threaded members 25b having segmented areas 25c. The upper portion 24 has a base 24a and two upright members 24b cooperatively connected thereto. The top surface of the base 24a has a corrugated area 24c.

The second embodiment of the stabilization element, designated as 26 has a corrugated upper surface 26a and a corrugated bottom surface 26b. The corrugated surfaces 26a, 26b and 24c could also be described as having a vertical toothed surface. The toothed surface 26b meshes with the toothed surface 24c, thereby preventing relative movement between the stabilization element 26 and the bone screw 23. With such a vertical tooth configuration, it is not necessary to create an opening in the stabilization element 26. The uprights 24b are spaced to have a distance between them the approximate width of the stabilization element 26. Therefore the upright members 24b also assist in limiting relative movement of the stabilization element 26 to the bone screw 23.

A preferred embodiment of a stabilization device 30 is shown in FIGS. 12 through 14. The device 30 includes a stabilization means 32 and an anchoring means 34. As shown in these Figures, the stabilization means 32 and the anchoring means 34 are of unitary construction. The anchoring means 34 are preferably elongate and extend from the stabilization means 32 at approximately 90 degrees to a longitudinal axis 36 of the stabilization means 32. As shown in FIG. 14, the stabilization means and anchoring means may be channeled out to form a lumen axially disposed therethrough. Alternatively, only the anchoring means may include the lumen. The lumen promotes additional ingrowth of bone in accordance with the method of the present invention as set forth in detail below.

Preferably, the stabilization device 30 is constructed and arranged for the specific stabilization task it will perform. For example, if it is desired to stabilize three vertebrae, a pair of devices 30 will be designed to include three anchoring means 34 each. The individual posts of the anchoring means will be spaced apart to match the spacing of the pedicles of the vertebrae. If only two vertebrae are to be stabilized, such as is shown in FIG. 12, a pair of devices 30 are constructed to have two posts 34 extending from the stabilization means 32, which are spaced apart to match the pedicles 11a and 12a, or 11b and 12b. The holes 14 are drilled in the pedicles 11 and 12 to precisely match the positions of the posts 34.

The method of the present invention begins after a determination is made that two or more vertebrae, 11 and 12, require stabilization and the appropriate vertebrae, 11 and 12, have been identified. Appropriate initial steps are taken to expose the spine and control the bleeding, thereby creating a suitable working environment for the procedure. Next, a posterior element 13 (FIG. 9) from a first vertebra 11 is removed, thereby exposing a first and second vertebral pedicle, 11a and 11b respectively, on the first vertebra 11. These pedicles 11a and 11b will later serve as the device anchoring point for that vertebra 11. Similarly, the next step is to remove a posterior element 13 from a second vertebra 12, adjacent the first vertebra 11, thereby exposing a first and second vertebral pedicle, 12a and 12b respectively, on the second vertebra 12. This is repeated for all of the targeted vertebrae, though for purposes of this example, only two vertebrae, 11 and 12, will be targeted. Notably, the method of the present invention lends itself readily to nerve decompression. Removal of all or the medial inferior portion 38 of the pedicles relieves pressure placed on swollen nerves by the pedicles.

Next, holes 14 must be formed in the first and second vertebral pedicles, 11a and b, and 12a and b, on the first and second vertebrae 11 and 12. Care is taken to accurately measure the spacing between the holes 14, which should closely match the distance between the centers of the posts 34 of a pre-selected stabilization device 30. The diameter of the holes 14 should exceed the diameter of the posts 34, thereby providing room for error as well as room for the existence of an activation slurry, which will be placed into the holes 14 with the posts 34 and promote an integration of the vertebrae 11 and 12.

The next step is to create the activation slurry (not shown), if osteoblastic induction is chosen as an external activation method. This slurry is created by milling autogenous bone, local or distal. Preferably the removed posterior elements are used, thereby obviating the need for a separate harvesting surgery. The milled bone is combined with autogenous stem cell or bone morphogenic materials. The slurry is then placed in the hole 14 to serve as an osteoblast resource.

If fibroblastic induction is chosen as an external activation method, autogenous fat grafts (local or distal, but preferably local, again obviating the need for a second harvesting operation) are placed in proximity to the microporous components, such as in the hole 14, around the post 34, or near the union of the device 30 and the vertebrae 11 and 12.

The vertebrae 11 and 12 are now prepared to receive the stabilization device 30. The anchoring means or posts 34 of the device 30 are simply placed into the holes 14, giving appropriate deference to the preservation of the fat grafts, if any. A first stabilization device 30a is placed on one side of the vertebrae and a second stabilization device 30b is placed on the other side, as shown in FIG. 12.

Measures are next taken to secure the device 30 in place until the slurry has a chance to harden and grow. Acceptable measures include applying polymers or cements. These substances may be self-curing or may require curing by irradiation, such as infrared, or thermal curing.

Other modifications of the invention will be apparent to those skilled in the art in light of the foregoing description. This description is intended to provide specific examples of individual embodiments, which clearly disclose the present invention. Accordingly, the invention is not limited to these embodiments or the use of elements having specific configurations and shapes as presented herein. All alternative modifications and variations of the present invention that follow in the spirit and broad scope of the appended claims are included.

1. A device useable to connect adjacent vertebrae comprising:
a stabilization member;
a first anchoring member extending from the stabilization member; and,
a second anchoring member extending from the stabilization member;
said stabilization member, said first anchoring member, and said second anchoring member, being of unitary construction.

2. The device of claim 1 wherein said stabilization member is porous.

3. The device of claim 1 wherein said first anchoring member extends perpendicularly from the stabilization member.

4. The device of claim 1 wherein said second anchoring member extends perpendicularly from the stabilization member.

5. The device of claim 1 wherein said first and second anchoring members are parallel to each other.

6. The device of claim 1 wherein at least one of said first and second anchoring members is substantially cylindrical.

7. The device of claim 1 wherein at least one of said first and second anchoring members is porous.

8. A method of stabilizing intervertebral joints comprising:
removing a posterior element from a first vertebra, thereby exposing a first and second vertebral pedicle on the first vertebra;
removing a posterior element from a second vertebra, adjacent the first vertebra, thereby exposing a first and second vertebral pedicle on the second vertebra;
removing medial inferior portions of the exposed first and second vertebral pedicles;
forming holes into the first and second vertebral pedicles on the first and second vertebrae;
providing a first and second device useable to connect adjacent vertebrae, each device having:
a stabilization member;
a first anchoring member extending from the stabilization member; and,
a second anchoring member extending from the stabilization member;
said first and second anchoring members comprising a biocompatible material capable of accepting an ingrowth of organic material; and,
placing the first anchoring member of the first device in the hole formed in the first pedicle of the first vertebra;
placing the second anchoring member of the first device in the hole formed in the first pedicle of the second vertebra;
placing the first anchoring member of the second device in the hole formed in the second pedicle of the first vertebra;
placing the second anchoring member of the second device in the hole formed in the second pedicle of the second vertebra; and,
activating local cellular proliferation.

9. The method of claim 8 wherein activating local cellular proliferation comprises:
forming a slurry having stem cells using one or both of the removed posterior elements;
placing a quantity of the slurry in each of the holes.

10. The method of claim 8 wherein activating local cellular proliferation comprises externally activating the anchoring members.

11. The method of claim 10 wherein externally activating the anchoring members comprises placing a slurry of milled autogenous bone and autogenous stem cell materials between the vertebrae and the devices to serve as an osteoblast resource.

12. The method of claim 10 wherein externally activating the anchoring members comprises placing a slurry of milled autogenous bone and bone morphogenic materials between the vertebrae and the devices to serve as an osteoblast resource.

13. The method of claim 10 wherein externally activating the anchoring members comprises placing autogenous fat grafts in proximity to the devices to serve as a resource for fibroblasts.

14. The method of claim 8 further comprising fastening the devices to the vertebrae to prevent relative movement therebetween while ingrowth proliferates.

15. The method of claim 14 wherein fastening the devices to the vertebrae to prevent relative movement therebetween while ingrowth proliferates comprises adhering the devices to the vertebrae using an adhesive.

16. The method of claim 15 wherein using an adhesive comprises placing a cement between the devices and the vertebrae.

17. The method of claim 15 wherein using an adhesive comprises placing a polymer between the devices and the vertebrae.

18. The method of claim 15 wherein using an adhesive comprises placing an adhesive between the devices and the vertebrae and curing the adhesive.

19. A method of stabilizing intervertebral joints comprising:
removing a posterior element from a first vertebra, thereby exposing a first and second vertebral pedicle on the first vertebra;
removing a posterior element from a second vertebra, adjacent the first vertebra, thereby exposing a first and second vertebral pedicle on the second vertebra;
removing all of the exposed first and second vertebral pedicles leaving a footprint of the removed pedicles;
forming holes into the first and second vertebral bodies at the pedicle footprint;
providing a first and second device useable to connect adjacent vertebrae, each device having:
a stabilization member;
a first anchoring member extending from the stabilization member; and,
a second anchoring member extending from the stabilization member;
said first and second anchoring members comprising a biocompatible material capable of accepting an ingrowth of organic material; and,
placing the first anchoring member of the first device in the hole formed in the first vertebral body at the first pedicle footprint;
placing the second anchoring member of the first device in the hole formed in the second vertebral body at the first pedicle footprint;
placing the first anchoring member of the second device in the hole formed in the first vertebral body at the second pedicle footprint;
placing the second anchoring member of the second device in the hole formed in the second vertebral body at the second pedicle footprint; and,
activating local cellular proliferation.

20. The method of claim 19 wherein activating local cellular proliferation comprises:
forming a slurry having stem cells using one or both of the removed posterior elements;
placing a quantity of the slurry in each of the holes.

21. The method of claim 19 wherein activating local cellular proliferation comprises externally activating the anchoring members.

21. The method of claim 20 wherein externally activating the anchoring members comprises placing a slurry of milled autogenous bone and autogenous stem cell materials between the vertebrae and the devices to serve as an osteoblast resource.

22. The method of claim 20 wherein externally activating the anchoring members comprises placing a slurry of milled autogenous bone and bone morphogenic materials between the vertebrae and the devices to serve as an osteoblast resource.

23. The method of claim 20 wherein externally activating the anchoring members comprises placing autogenous fat grafts in proximity to the devices to serve as a resource for fibroblasts.

24. The method of claim 19 further comprising fastening the devices to the vertebrae to prevent relative movement therebetween while ingrowth proliferates.

25. The method of claim 24 wherein fastening the devices to the vertebrae to prevent relative movement therebetween while ingrowth proliferates comprises adhering the devices to the vertebrae using an adhesive.

26. The method of claim 25 wherein using an adhesive comprises placing a cement between the devices and the vertebrae.

27. The method of claim 25 wherein using an adhesive comprises placing a polymer between the devices and the vertebrae.

28. The method of claim 25 wherein using an adhesive comprises placing an adhesive between the devices and the vertebrae and curing the adhesive.

29. The method of claim 19 wherein said biocompatible material capable of accepting an ingrowth of organic material is a porous material.

30. The method of claim 19 wherein said first and second anchoring members include a lumen axially disposed therethrough integral flexible spine stabilization device and method.
